(19) United States
(12) Patent Application Publication
(10) Pub. No.: US 2023/0038250 A1
(43) Pub. Date: Feb. 9, 2023

(54) TEMPORARY COSMETIC HAIR FOLLICLE MICROPIGMENTATION SYSTEM AND METHOD

(71) Applicant: Michael Witt, San Juan, PR (US)

(72) Inventor: Michael Witt, San Juan, PR (US)

(21) Appl. No.: 17/883,558

(22) Filed: Aug. 8, 2022

Related U.S. Application Data
(60) Provisional application No. 63/231,062, filed on Aug. 9, 2021.

(51) Int. Cl. A61K 8/92; A61K 8/02; A61K 8/96; A61Q 7/00

(52) U.S. Cl. CPC A61K 8/92; A61K 8/027; A61K 8/96; A61Q 7/00; A61K 2800/43

(57) ABSTRACT

A cosmetic product, system and method that creates a temporary, semipermanent, stubble-like appearance to bald or balding areas of the human head and face where facial hair typically grows. The bald or balding areas are covered in an oil to accept a pigment material, such as activated charcoal, applied to a desired area. The pigment material is then covered with a sealing agent and waterproofing agent to prevent smudging and enable the user to expose the covered area to water without losing the effect.

### TECHNICAL FIELD

The present description relates to a cosmetic product, system and method that creates a temporary, semi-permanent, stubble-like appearance to bald or balding areas of the human head, and/or to the face where men are not able to grow hair, and/or if a female wanted to give the appearance of having facial hair. More particularly, the description relates to a cosmetic product that gives the appearance of stubble by binding to both hair follicles located on the scalp where hair once grew, but is now either microscopic or no longer grows, and on the epidermis of the scalp or face where a beard would naturally occur.

### BACKGROUND

Androgenic alopecia, the medical term for the inherited form of hair loss, also known as male-pattern baldness (MPB) and female-pattern baldness (FPB), is, in the vast majority of cases, caused by inherited genetic factors.

While much has been discovered as to the cause of MPB and FPB, such as the amounts of specific hormones produced, such as dihydrotestosterone, and the hair follicle's susceptibility and sensitivity to them, to date there is not a cure for MPB or FPB.

A hair follicle is an organ that looks like a tunnel-shaped structure. Hair shafts emerge from a hair follicle through the upper layer of the skin called the epidermis and through the top of the hair follicle called the pore. The hair follicle's pouch-like structure runs from the skin's surface, or epidermis, down into the dermis or dermal layer and consists of many parts, including the hair papilla, hair matrix, hair bulb, apocrine sweat gland, sebaceous gland, and the arrector pili muscle.

Hair begins to grow at the bottom of the hair follicle. The root of an individual hair is made up of protein cells called keratin that are fed by blood vessels close by, creating more cells making the hair grow out of the follicle and up out of the skin through the pore. It is well known that during male pattern balding, the hair follicles begin to shrink over time, making the hair shafts progressively smaller. The number of follicles that shrink and amount of time it takes for the follicle to shrink, whether months or years, vary from case to case. Ultimately the hair shafts and hair follicles don't disappear, but shrink to such a small size as to be only visible with a microscope, with the hair shafts produced no longer being visible. Contrasted with the area of the scalp where hair continues to grow, the affected area with shrunk follicles gives the appearance of baldness. Individuals with completely bald scalps still have approximately 100,000 hair follicles that continue to make microscopically visible hairs that often do not reach the surface of the skin.

Male-pattern baldness and female-pattern baldness typically follow a pattern, and while hair loss and follicle shrinkage can begin in different areas, in men thinning typically starts at the crown or temple and progresses, leaving the front, top and rear of the head bald in what has been referred to as a "horse-shoe" pattern.

With the advent of social media and the importance of image and ease of finding individuals by their name and image on the internet there has been an increase in societal pressures for individuals to maintain healthy, youthful appearances. Digital images become critical as an individual creates a professional profile on LinkedIn, a social network on the internet designed for career and business professionals to connect. The same can be said for romantic connections on ubiquitous dating apps. It is now common for individuals to seek serious romantic relationships much later in life. It is common for men to seek partners while in their thirties which makes MPB more of a problem with increased likelihood compared to previous generations the older they become. Men losing their hair due to MPB have described the impact on their confidence as losing pieces of their identity little by little as they became bald. Studies have shown the detrimental impact on the mental health of both men and women and the direct link to increased anxieties and mental health conditions with both male and female pattern baldness. Men and women with male and female pattern baldness claim to feel less attractive which directly impacts their confidence, creating social angst and nervousness how they are perceived by others.

Statistics show that male pattern baldness impacts 50% of all men over the age of fifty, and 50% of women over the age of sixty-five. In the United States, there is an increasing trend for men to shave their heads as they begin to lose their hair. Due to Hollywood icons such as Bruce Willis, Dwayne Johnson, Vin Diesel and Jason Statham, all who have shaved heads, there is now a perception that men with shaved heads emanate dominance, authority, strength, leadership ability and being in control, with an overall tougher image.

Research studies performed by Dr. Albert Mannes, a lecturer at the Wharton School of the University of Pennsylvania, support these findings as he tested people's perceptions of men with shaved heads (complete findings published in the journal "Social Psychological and Personality Science"). In conclusion, the studies found that men with shaved heads were perceived as powerful, influential, authoritative, strong and linked with dominance more than men with full heads of hair. The overall perception backed by the research showed that men with shaved heads were more confident and masculine. The research studies found that men with a full head of hair scored only slightly higher only in attractiveness and confidence than men with shaved heads. While men with regular-length, thinning hair scored the lowest on every trait in the study, except for "norm violation." One of the conclusions Dr. Mannes came to was that balding men could benefit their self-esteem and perception of themselves with others by shaving their head. Finally, the study showed that men who shaved their heads looked on average four years older than they were.

While men with shaved heads inadvertently benefit from their look, given the option to have a full head of hair with a cure that currently does not exist, a majority of men said they would prefer to have a full head of hair. Men and women spend annually worldwide over a billion dollars on Minoxidil alone, a treatment that offers only marginal results in slowing the rate of hair loss in men and women. Finasteride is another drug used to stop baldness and regrow hair that offers marginal benefits while introducing other side-effects and problems such as erectile dysfunction in some men as the drug reduces male hormone activity. A drug capable of stopping baldness and proving full effectiveness to regrown hair to date has not been developed.

Plastic surgery in the form of hair transplants has been a cosmetic option as the doctor takes hair from the thicker parts of the scalp and grafts it into the thinning or balding sections of the scalp. While hair transplants have proven more successful than over-the-counter drugs, such as Minoxidil and Finasteride, there is a wide margin, between 10%-80% of hair follicles that will produce hair in the months following the surgery. Transplanted hair, like the hair that it replaced, will also face the likelihood of thinning with passing of time. Hair transplants cost on average between USD $4,000 \-$15,000 per session. The expensive procedure is not guaranteed to work and may leave scaring. Other common side effects of hair transplants are infection of hair follicles, scalp swelling, and itchiness. There is also a chance that the transplanted hair will be lost, referred to as "shock loss." If a cure for baldness is found, the naturally occurring hair follicles that would be able to regenerate hair will have been removed due to the hair transplant procedure. Finally, hair transplants in an ongoing treatment, usually requiring multiple sessions as male and female pattern baldness is progressive with ever increasing spread over the scalp.

Hair loss due to male and female pattern baldness is viewed worldwide as a cosmetic problem, not as a disease. Because of this, both industry and academic funding has been minimal in comparison to what is spent on disease. While consumer demand for solutions to MPB and FPB has been proven by the amount of money spent currently on Minoxidil and Finasteride markets, medical industries are not willing to spend large amounts of money given that health insurance companies are not likely to cover their patients should a drug be created that cures male and female pattern baldness.

Scalp micropigmentation is currently one of the fastest-growing, hair-loss solutions in the United States. This treatment does not stop hair loss or stimulate new hair growth. Micro needles are used to deposit tiny dots of pigment into the dermal layer of the skin on the scalp to create the appearance of a full head of naturally occurring shaved hair in the form of stubble. The average cost is between USD $1800-$3200 and the pigmentation lasts between 3-5 years depending on various factors. A person's immune system sees the pigmentation as a foreign invader and attacks it, causing it to shrink and fade over time, necessitating the need for future sessions to maintain the correct pigment shade. The sun's UV light and exposure to it causes the micropigmentation ink to fade faster and the need for more subsequent touch-up sessions. The process of the micro needling causes mild discomfort as some areas of the scalp have more, denser nerve endings than other parts. Due to the wide variation in the amount of training, skill and expertise of the individual doing the micropigmentation from company to company, the quality of the micropigmentation and how real the stubble and shading looks can vary from very real, to very poorly done with large discolored spots and is easy to tell that the micropigmentation is not real hair due to the discoloring, fading, dots that are too large to be the size of real hair, and that the frontal areas of the scalp being the same darkness as the sides, whereas in real life, these hairs on most men would start to shrink, giving a fading affect. Other downsides to permanent micropigmentation include the hairline being tattooed on too low. The major downside of this hair-loss solution is that the results may not be as expected and cannot be easily changed due to the permanent nature. It has been common that technicians apply the pigment in a way to give a monochromatic appearance, or that of a "skin helmet." Other times, technicians have applied the micropigmentation too deeply into the client's skin giving off a blue or green tint that does not look like hair. The ink dots may begin to connect overtime as the ink dots bleed into each other.

### BRIEF SUMMARY OF THE INVENTION

According to an embodiment, a method is provided for providing semi-permanent micropigmentation that adheres to low functioning hair follicles and the scalp, but is not limited to the epidermis found on the scalp, leaving micro-dots so as to give the appearance of stubble-like hair in areas where the hair either has stopped growing, such as with male and female pattern baldness.

A user, or other person, applies a carrier oil, such as, but not limited to Castor Oil, to a desired area, e.g. the user's scalp, and then applies a micronized powder of activated charcoal to attach to the hair follicle pores on the scalp and adhere to low functioning hair follicles and the skin around the low functioning follicles on such areas, but not limited to the epidermis found on the scalp, leaving micro-dots of color and on around the pores where hair used to grow so as to give the appearance of stubble-like hair in areas where the hair has stopped growing. In another embodiment, another pigment providing material is used, such as a hair fiber builder.

In an embodiment, the carrier oil and micronized powder of activated charcoal are mixed to enable simultaneous application.

To provide stability and maintain the effect, a waterproofing agent, such as waterproof sunscreen, may be applied. A sealing agent, such as hairspray may be applied to prevent smudging and offer waterproofing . The mixed oil and charcoal may be waterproof, but not touch proof. To make touch resistant, the next step may be to spray with hairspray, but then the stubble may lose some of its inherent waterproof quality. To get it back, a further step would be to then use spray on sunscreen. It then is both touch resistant and waterproof.

In an embodiment, the functional chemicals in the sunscreen and hairspray, i.e., those chemicals that provide the desired properties of sun protection, waterproofing, and sealing, may be combined into a solution, thereby eliminating the need for two products.

The many advantages of the end product is that it creates the illusion of human hair stubble. It is easily and painlessly applied and easily removed by washing off with soap and water.

### BRIEF DESCRIPTION OF THE DRAWINGS

FIG. 1 is a flowchart describing a an exemplary embodiment of a method for applying a temporary cosmetic hair follicle treatment.

FIG. 2 is a perspective view of a pigment material application.

FIG. 3 is a perspective view of a waterproofing material application.

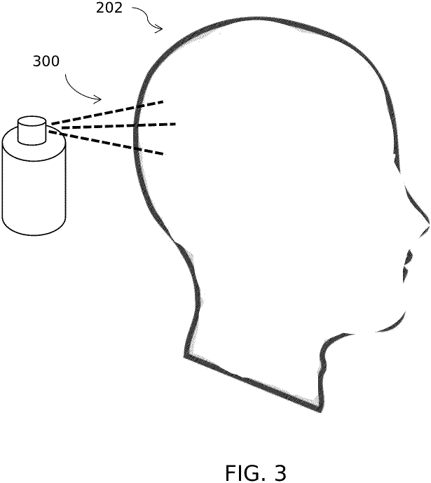

### DETAILED DESCRIPTION

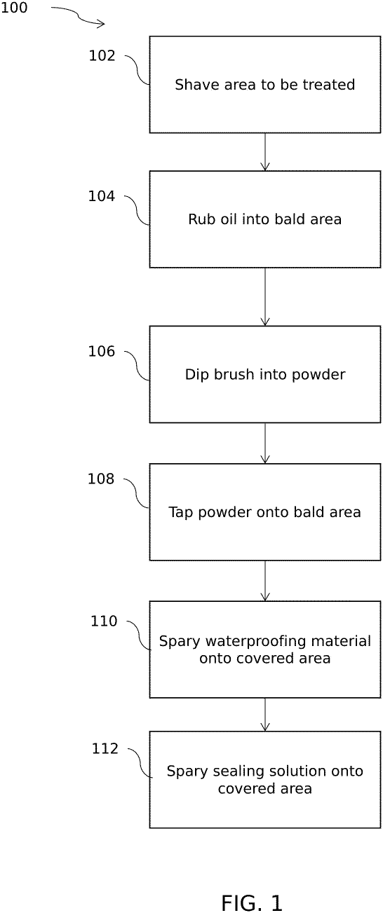

FIG. 1 is a flowchart showing an exemplary embodiment of a method 100 for applying a temporary cosmetic hair follicle treatment. The user shaves 102 all the remaining hair on their head so as to give as much uniformity to the area of the scalp where hair no longer grows to that area where hair still grows.

The user then rubs 104 a small amount of carrier oil on the bald area of the scalp. There may be overlap onto areas of the scalp where hair is still naturally growing and or, the entire scalp. Any number of carrier oils can be used, such as but not limited to Argan oil, Castor oil, Moringa oil, Coconut oil or Jojoba oil.

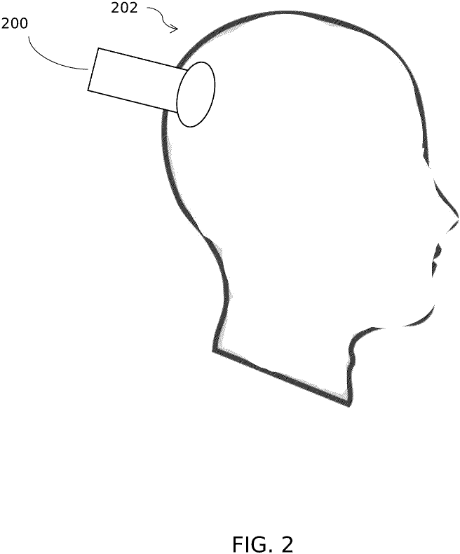

The user dips 106 the brush into a loose powder of the micronized activated charcoal powder. The brush 200, shown in FIG. 2, could be a loose powder brush commonly used with women's cosmetics, a powder puff, a make-up blending sponge, or any number of brushes currently on the market including but not limited to paint brushes. The user then taps the powder 106 onto the bald area of the scalp and brushes it over the scalp 202 making a fine uniform covering, as shown in FIG. 2.

Because of the dark nature of the micronized activated charcoal powder, it is easy for the user to be able to spread across the bald stretches of skin to the dark areas where the shaved hair follicles where hair is still growing begins. The user can also brush it over the sides and back where stubble is still naturally occurring to create a more uniform look.

In an embodiment, a shake-on concealers called hair-building fibers may be used instead of activated charcoal. These shake on concealers are made of plant-based hair building fibers that are shaken onto the consumer's head and cling to existing hairs with natural, strong static charge that creates a magnetic effect, binding the fibers to existing strands of hair, giving thinning areas a fuller look. There are many different companies offering their own hair-building plant-fiber compositions, such as wool, Keratin and cotton. Hair building fibers may come in different colors to match the color of the user's natural hair.

In an embodiment, the pigment material, e.g., activated charcoal or hair-building fibers, may be combined into the mixture, eliminating the need for the user to first rub the essential oil onto the scalp.

In order to prevent potential smudging of the pigment material, the user may spray 112 on a sealing solution, for example a high hold hair spray.

In an embodiment, the user may spray 110 a waterproof sunscreen 300 onto the covered area, e.g., the area on which the pigment material has been applied after it has been sprayed with the hairspray seal. This provides the dual benefit of providing protection from the sun and waterproofing the covered area. The enables the user to spend more time in the sun and enter the water and completely submerge without losing the effect.

In an embodiment, the functional chemicals in the sunscreen and hairspray, i.e., those chemicals that provide the desired properties of sun protection, waterproofing, and sealing, may be combined into a solution, thereby eliminating the need for two products. These chemicals include, but are not limited to:
Sunscreen:
   oxybenzone, avobenzone, octisalate, octocrylene, homosalate, octin-oxate, etc.
Waterproof sunscreen: waxes, oils, polymers such as dimethicone, etc.
Hair spray: polymers such as polyvinylpyrrolidone, polydimethylsiloxane, vegetable gum, etc.

The appropriate chemicals may be added in solution and sprayed on simultaneously.

The foregoing method descriptions and diagrams/figures are provided merely as illustrative examples and are not intended to require or imply that the operations of various aspects must be performed in the order presented. As will be appreciated by one of skill in the art, the order of operations in the aspects described herein may be performed in any order. Words such as "thereafter," "then," "next," etc. are not intended to limit the order of the operations; such words are used to guide the reader through the description of the methods and systems described herein. Further, any reference to claim elements in the singular, for example, using the articles "a," "an," or "the" is not to be construed as limiting the element to the singular.

The preceding description of the disclosed aspects is provided to enable any person skilled in the art to make, implement, or use the claims. Various modifications to these aspects will be readily apparent to those skilled in the art, and the generic principles defined herein may be applied to other aspects without departing from the scope of the claims. Thus, the present disclosure is not intended to be limited to the aspects illustrated herein but is to be accorded the widest scope consistent with the claims disclosed herein.

What is claimed is:

1. A method for applying a cosmetic product that temporarily creates the appearance of stubble, the method comprising:
applying an oil on a user's skin;
applying a pigment material to a desired area on the oil covered area on the user's skin;
applying a sealing agent to the covered area; and
applying a waterproofing agent to the covered area.

2. The method of claim 1, wherein the desired area is the user's scalp.

3. The method of claim 1, wherein the pigment material comprises activated charcoal.

4. The method of claim 1, wherein the pigment material comprises a hair fiber builder.

5. The method of claim 1, wherein the waterproofing agent comprises waterproof sunscreen.

6. The method of claim 1, wherein the sealing agent comprises hairspray.

7. The method of claim 1, wherein said applying the oil and applying the pigment material comprises applying a mixture of the oil and the pigment material such that said applying the oil and applying the pigment material are performed simultaneously.

8. The method of claim 1, wherein said applying the waterproofing agent and applying the sealing agent comprises applying a solution including a mixture of the functional chemicals in the waterproofing agent and sealing agent such that said applying the waterproofing agent and applying the sealing agent are performed simultaneously.

9. The method of claim 1, wherein the pigment material comprises a mixture of activated charcoal and hair fiber builder.
